(19) United States
(12) Patent Application Publication
(10) Pub. No.: US 2008/0171729 A1
(43) Pub. Date: Jul. 17, 2008

(54) Use Of A Steroid For Enhancement Of Skin Permeability

(75) Inventor: Josef Gottfried Meingassner, Perchtoldsdorf (AT)

Correspondence Address: MONTGOMERY, MCCRACKEN, WALKER & RHOADS, LLP, 123 SOUTH BROAD STREET, AVENUE OF THE ARTS, PHILADELPHIA, PA, 19109, US

(73) Assignee: Novartis AG

(21) Appl. No.: 11/571,895

(22) PCT Filed: Jul. 15, 2005
(86) PCT No.: PCT/EP05/07740

§ 371 Date: Sep. 20, 2007

(30) Foreign Application Priority Data
Aug. 2, 2004 (GB) 0417205.2
Aug. 27, 2004 (GB) 60605328
Sep. 16, 2004 (GB) 0415994.3

(51) Int. Cl. A61K 31/573; A61P 17/06; A61P 35/00; A61K 31/407; A61K 31/4375; A61K 31/395; A61P 37/02

(52) U.S. Cl. 514/171; 514/183; 514/411; 514/291; 514/182

(57) ABSTRACT

A method for enhancing skin permeability by administration of a tetracyclic compound and improved methods for treatment of disorders by administration of a tetracyclic compound and a pharmaceutically active compound.

The present invention relates to the use of tetracyclic compounds, such as steroids, for the enhancement of skin permeability, e.g. for use in the enhancement of skin permeability for a pharmaceutically active compound, such as a compound for therapeutic use.

Tetracyclic compounds, such as steroids, e.g. including corticoids, may exhibit numerous pharmaceutical activities. We have now found surprisingly an unknown use of such tetracyclic compounds.

In one aspect the present invention provides a tetracyclic compound for use in the enhancement, e.g. improvement, of skin permeability.

According to the present invention, for enhancing skin permeability a tetracyclic compound is administered preferably topically (epicutanously).

In another aspect the present invention provides a tetracyclic compound for topical (epicutanous) use in the enhancement, e.g. improvement, of skin permeability.

Enhanced skin permeability as used herein means that the infiltration of a pharmaceutically active compound other than a tetracyclic compound according to the present invention, e.g. when administered topically (epicutanously), into the skin and subcutaneous tissue is enhanced, e.g. improved, such as accelerated, if the skin is treated with a tetracyclic compound, compared with the permeability for a pharmaceutically compound of untreated skin (e.g. skin which is not treated with a tetracyclic compound according to the present invention). Treatment includes simultaneous treatment and pre-treatment, preferably pre-treatment.

In another aspect the present invention provides a tetracyclic compound for, e.g. topical (epicutanous), use for the enhancement, e.g. improvement, such as acceleration, of the skin permeability for a pharmaceutically active compound, which pharmaceutically active compound is other than a tetracyclic compound according to the present invention.

We also have found, that the skin permeability for pimecrolimus when administered topically (epicutanously) may be enhanced if the skin is treated, e.g. pre-treated, with a tetracyclic compound.

In another aspect the present invention provides a tetracyclic compound for, e.g. topical (epicutanous), use in the enhancement, e.g. improvement, such as acceleration, of the skin permeability for a compound of formula I, such as of formulas I_{P}.

The barrier function of the skin plays a pivotal role in the percutaneous absorption of epicutaneously applied drugs. In a side-by-side comparison we have evaluated the penetration (infiltration into the epidermal and superficial dermal layers) and permeation (infiltration across superficial layer into deep dermal layers) of pimecrolimus and tacrolimus in untreated and corticosteroid (CS) treated (pre-treated) porcine skin, wherein pimecrolimus and tacrolimus are applied epicutaneously as the marketed formulations (Elidel 1% cream, Protopic 0.1% ointment). We have found that the skin permeability for pimecrolimus and tacrolimus are enhanced when skin is pre-treated with CS; e.g. we have found the skin permeability to be increased by factors of 3.6 (pimecrolimus) and 1.7 (tacrolimus) as compared with normal untreated skin. These factors have been found to be practically independent of the pharmaceutical, e.g. therapeutical, activity (potency) of the CS used because of comparable permeation rates in case of the weakly potent hydrocortisone with the high potent clobetasol. We therefore estimate that the chemical structure of a tetracyclic compound, e.g. as defined below, may be responsible for high skin permeation. High skin permeation may result in efficacious concentrations of the active compound in affected epidermal and dermal layers.

The present invention will facilitate skin permeability of e.g. T cell modulators to be effective against pathogenic T cells in skin layers, e.g. dermal layers, infiltrated with pathogenic T cells. In other words, a tetracyclic compound when administered before or simultaneously, preferably before, administering a pharmaceutically active compound, may enhance the skin permeability for such pharmaceutically active compound compared with the skin permeability of untreated skin and, if the pharmaceutically active compound is of formula I, such as of formula I_{P}, also skin penetration by such compound may be enhanced. According to the present invention a pharmaceutically active compound is other than a tetracyclic compound of the present invention. If administered topically (epicutanously) a tetracyclic compound is administered to that part of the skin to which a pharmaceutically active compound is intended to be administered.

The consequence of such (pre-)treatment with a tetracyclic compound may be that
the infiltration into the skin of a pharmaceutically active compound is enhanced, e.g. accelerated, and
the pharmaceutically active compound may infiltrate deeper into the skin, e.g. deeper into the dermal layers.
   compared with administration of said pharmaceutically active compound to untreated skin, i.e. administration to skin which is not treated with a tetracyclic compound according to the present invention.

In another aspect the present invention provides a method of enhancing the skin permeability for a pharmaceutically active compound comprising treating the skin with a tetracyclic compound and administering a pharmaceutically active compound.

e.g. treating that part of the skin, where a pharmaceutically active compound is intended to be administered,
e.g. using an effective amount of a tetracyclic compound and an effective amount of a pharmaceutically active compound,
e.g. by topical (epicutanous), administration of the tetracyclic compound and of the pharmaceutically active compound, and
administering a pharmaceutically active compound, e.g. a compound of formula I, such as of formula Iₚ.

A pharmaceutically active compound according to the present invention includes pharmaceutically active compounds which effectively may be administered topically (epicutaneously) and which are permeating the skin for action in vivo. Such compounds e.g. include T-cell modulators, other than tetracyclic compounds according to the present invention, e.g. other than steroidal T-cell modulators.

In another aspect the present invention provides a tetracyclic compound
for use, or
in a method
   according to the present invention, wherein a pharmaceutically active compound is a non-steroidal T-cell modulator.

In a preferred aspect of the present invention a pharmaceutically active T-cell modulator is a calcineurin inhibitor.

In another aspect the present invention provides a tetracyclic compound
for use, or
in a method
   according to the present invention, wherein a pharmaceutically active compound is a calcineurin inhibitor.

Calcineurin is a calcium/calmodulin-regulated protein phosphatase involved in intracellular signalling. For reviews on calcineurin see e.g. Rusnak and Mertz, Physiol. Rev. 80, 1483-1521 (2000) and Feske et al., Biochem. Biophys. Commun. 311, 1117-1132 (2003).

Calcineurin inhibitors are substances which block calcineurin dephosphorylation of appropriate substrates.

A calcineurin inhibitor of the present invention is preferably an immunophilin binding compound having calcineurin inhibitory activity.

Immunophilin binding calcineurin inhibitors are compounds forming calcineurin inhibiting complexes with immunophilins, e.g. cyclophilin and macrophilin.

Examples of cyclophilin-binding calcineurin inhibitors include e.g. cyclosporins or cyclosporin derivatives (hereinafter cyclosporins). Cyclosporins and their preparation are e.g. disclosed in U.S. Pat. No. 4,117,118, wherein in a compound of formula I preferred substituents are indicated, which preferred substituents are also preferred substituents in the present application; e.g. in a compound of formula I each single defined substituent may be a preferred substituent, e.g. independently of each other substituent defined. Cyclosporin, originally extracted from the soil fungus Potypaciadium infilatum, has a cyclic 11-amino acid structure and includes e.g. Cyclosporins A through 1, such as Cyclosporin A, B, C, D and G, preferably Cyclosporin A.

In another aspect the present invention provides a tetracyclic compound
for use, or
in a method
   according to the present invention, wherein a pharmaceutically active compound is a cyclosporin, e.g. including a compound of formula

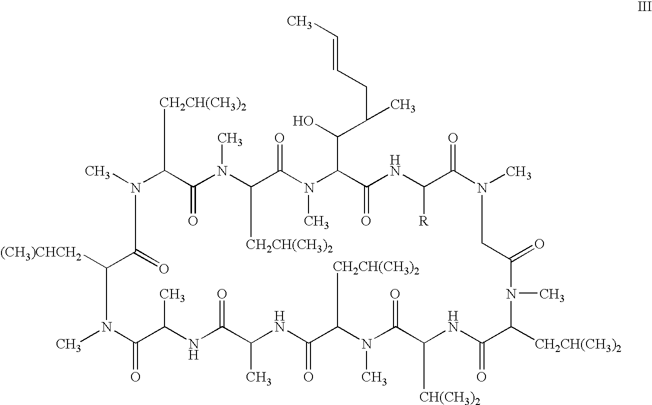

wherein R is methyl, ethyl, propyl, isopropyl or —CH(OH)CH₃, preferably R is ethyl (Cyclosporin A).

Examples of macrophilin-binding calcineurin inhibitors include ascomycin and ascomycin derivatives (hereinafter ascomycins), see e.g. Liu et al., Cell 66, 807-815 (1991) and Dumont et al., J. Exp. Med., 176, 751-780 (1992), as well as tacrolimus (FK506).

Ascomycins and their preparation are known. Ascomycin (FR 520) is a macrolide antibiotic disclosed e.g. in U.S. Pat. No. 3,244,592 and in EP349061, wherein in a compound of formula I preferred substituents are indicated, which preferred substituents are also preferred substituents in the present application; e.g. in a compound of formula I each single defined substituent may be a preferred substituent, e.g. independently of each other substituent defined. A wide range of ascomycin derivatives are known, which are either naturally occurring amongst fungal species or are obtainable by manipulation of fermentation procedures or by chemical derivatization.

In another aspect the present invention provides a tetracyclic compound
for use, or
in a method
   according to the present invention, wherein a pharmaceutically active compound is a compound of formula

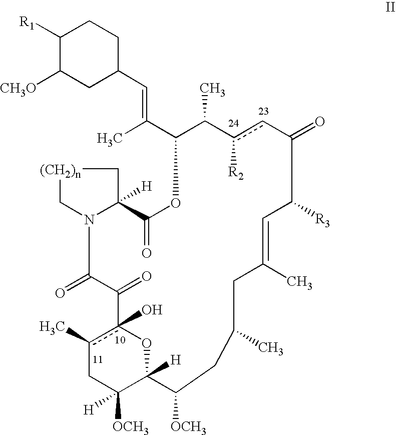

wherein
R₁ is hydroxy or protected hydroxy,
R₂ is hydrogen, hydroxyl or protected hydroxyl,
R₃ is methyl, ethyl, propyl or allyl,
n is an integer of 1 or 2, and the dotted line is a bond, or is no bond, preferably a compound of formula

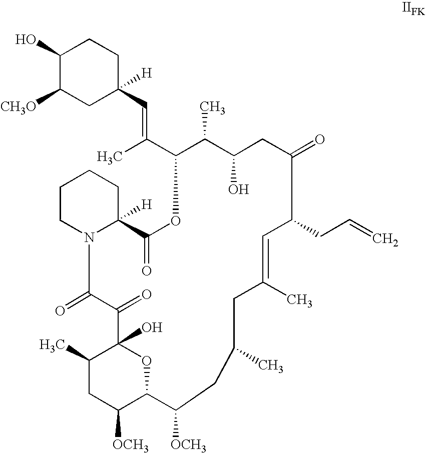

(tacrolimus, FK506).

If the dotted line is a bond, there is a double bond, if the dotted line is no bond there is a single bond in a compound of formula II, where a bond together with a dotted line is indicated.

In another aspect the present invention provides a tetracyclic compound
for use, or
in a method
   according to the present invention, wherein a pharmaceutically active compound is a compound of formula

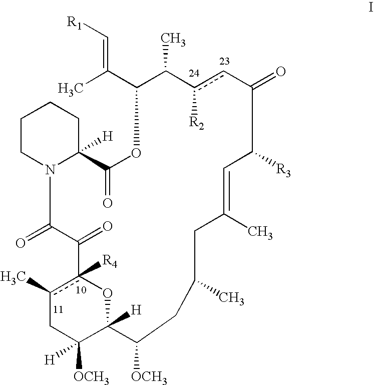

wherein either
R₁ is a group (a) of formula

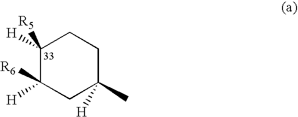

wherein
R₅ is chloro, bromo, iodo or azido,
R₆ is hydroxy or methoxy, and
R₄ is hydroxy and there is a single bond in 10,11 position; or absent, and there is a double bond in 10,11 position
   or
   R₁ is a group (b) or (c) of formula

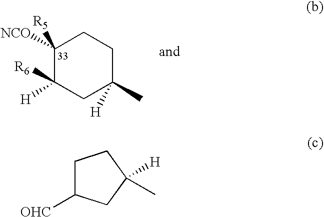

wherein
R₆ is as defined above, and
R₄ is hydroxy and there is a single bond in 10,11 position, R₂ is oxo and there is a single bond in 23,24 position; hydroxy and there is a single or double
bond in 23,24 position; or absent and there is a double bond in 23,24 position; and R₃ is methyl, ethyl, propyl or allyl, e.g. a compound of formula

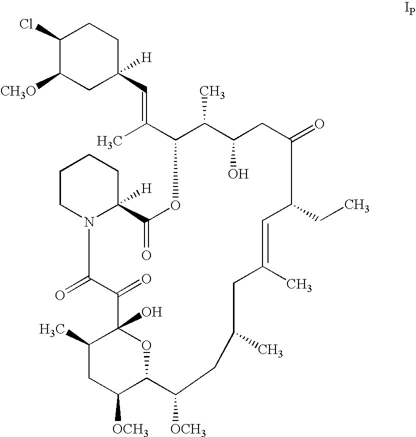

(pimecrolimus, ASM981).

Compounds of formula I are e.g. disclosed in EP-B-0427680, wherein in a compound of formula I preferred substituents are indicated, which preferred substituents are also preferred substituents in the present application; e.g. in a compound of formula I each single defined substituent may be a preferred substituent, e.g. independently of each other substituent defined; e.g. including a compound of formula I_{P}, such as disclosed in EP-B-0427680 in Example 66a as “33-epi-33-chloro-FR 520”, also known as “ASM981.

A tetracyclic compound as used herein includes a steroid, such as a compound having as a part of its chemical structure a group of formula

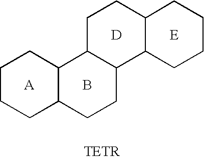

wherein each of the rings A, B, C and D have from 5 to 7 ring members,
e.g. ring A has 5 or 6 ring members,
e.g. ring B has 6 or 7 ring members,
e.g. ring D has 5 or 6 ring members,
e.g. ring E has 5 or 6 ring members.

Compounds of formula TETR preferably comprise compounds having as a part of its chemical structure

a. a group of formula

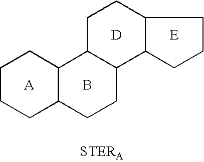

wherein rings A, B and D each have 6 ring members and E has five ring members,
   b. a group of formula

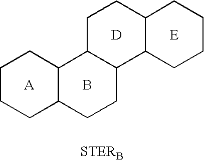

wherein rings A, B, D and E each have 6 ring members
   c. a group of formula

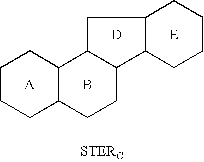

wherein rings A, B and E each have 6 ring members and D has five ring members, and
   d. a group of formula

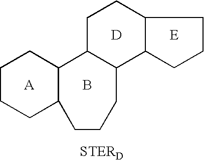

wherein rings A and D each have 6 ring members, B has 7 ring members and E has five ring members;
   more preferably of formula STER_{A}, STER_{B} or STER_{C}, and even more preferably of formula

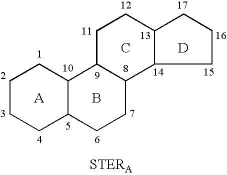

Ring members comprise carbon atoms and, preferably in rings A, B and E additionally hetero atoms selected from N, O, S, preferably from N or O. E.g. in ring A ring members include carbon atoms and heteroatoms selected from N or O, e.g. see dutasterin, samandarine. E.g. in ring B ring members include carbon atoms and heteroatoms selected from O, e.g. see brassinolide. E.g. in ring E ring members include carbon atoms and heteroatoms selected from 0, e.g. see cimigenol. E.g. in ring D all ring members are carbon atoms.

Preferably the ring members in rings A, B, D and E all are carbon atoms.

Rings A, B, D and E comprise saturated and partially or completely unsaturated, such as aromatic, rings.

Preferably

rings A and B comprise saturated and partially or completely unsaturated, such as aromatic rings, preferably
(C₅₋₇)cycloalkyl, (C₅₋₇)cycloalkylene, (C₅₋₇)cycloalkyldiene, wherein cycloalkyl preferably is hexyl; or phenyl; and
   rings D and E comprise saturated and partially unsaturated rings, preferably
(C₅₋₇)cycloalkyl, (C₅₋₇)cycloalkylene, (C₅₋₇)cycloalkyldiene, wherein cycloalkyl preferably is cyclopentyl or cyclohexyl.

Each of the rings A, B, D and E in a compound of formula TETR may be anellated with further rings, including rings having 3 to 8, preferably 3 to 6 ring members, which further rings may be anellated further with rings having 3 to 8, preferably 3 to 6 ring members, to form a ring system. Anellated rings (ring systems) include spiro-anellated rings. Anellated ring also include bridged rings, e.g. such as appropriate, e.g. such as conventional, e.g. including as a bridge

—O—, —CH₂—O—, —CH₂—NH—CH₂—CH₂—O— (e.g. see batrachotoxin).

Such anellated ring (systems) include saturated and partially and completely unsaturated ring (systems), optionally comprising heteroatoms selected from N, O and S, e.g. rings (ring systems) as described above.

Rings anellated with a ring system of formula TETR include for example
3-membered rings, e.g. including heterocyclic rings, such as cyclopropanes, oxiranes, thiiranes, e.g. of formulae

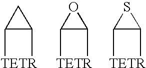

5-membered rings, e.g. including heterocyclic rings, such as
   5-membered rings comprising carbon atoms as ring members, such as cyclopentane, e.g. spiro-anellated with a further ring, e.g. of formula

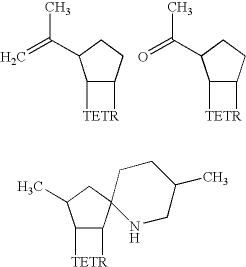

5-membered heterocyclic rings comprising one or more, e.g. two nitrogen atoms as heteroatoms, which ring may be anellated with a further ring, such as pyrrolidines, pyrazoles, including dihydropyrazoles, indolizines, such as octahydroindolizines, e.g. of formulae

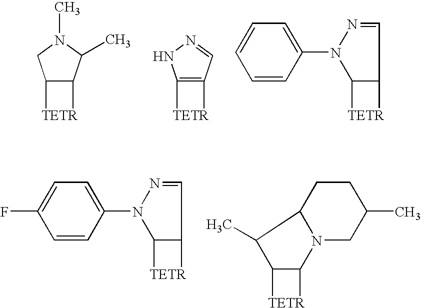

5-membered heterocyclic rings comprising one or two oxygen atoms, e.g. furanes, such as di- or tetrahydrofuranes, or 1,3-dioxolanes, e.g. of formulae

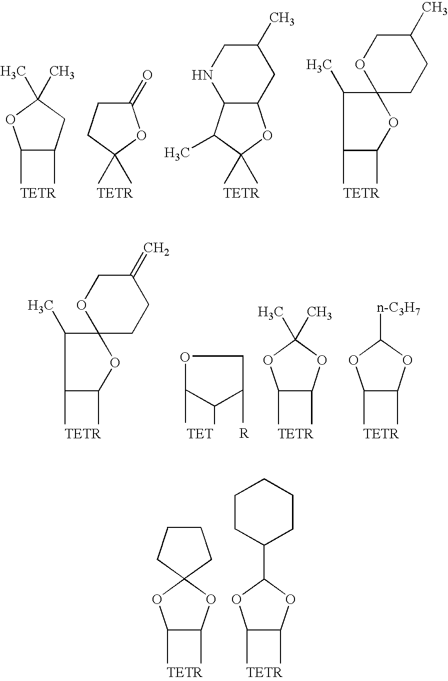

5-membered heterocyclic rings comprising oxygen atom and nitrogen atoms as hetero atoms, e.g. oxazoles, e.g. including dihydrooxazoles, isoxazoles, including dihydroisoxazoles, or furazanes, e.g. of formulae

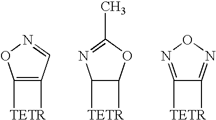

6-membered rings, e.g. including 6-membered rings comprising carbon atoms as the ring members and heterocyclic rings, e.g. comprising cyclohexanes and 6-membered heterocyclic rings comprising one or more heteroatoms selected from N, O or S, preferably N or O, such as
   cyclohexanes, e.g. of formula

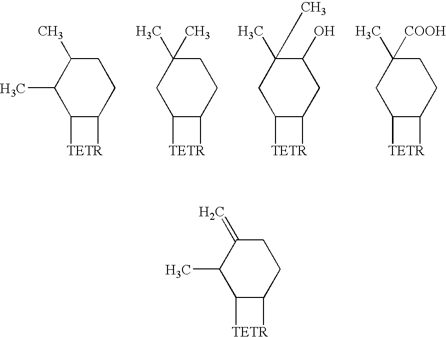

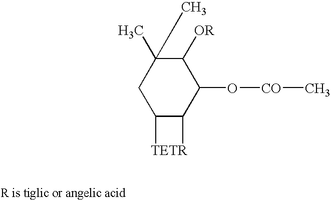

pyranes, such as tetrahydropyranes, e.g. bridged pyranes such as 6,8-dioxabicyclo[3.2.1]octane, and pyranes anellated with a further ring, such as octahydro-pyrano[3.2.b]pyridine, e.g. of formulae

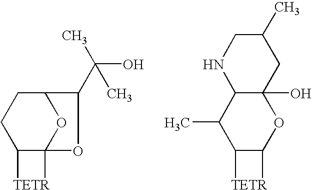

1,4-dioxanes, such as 1,4-dioxanes anellated with another ring, e.g. of formula

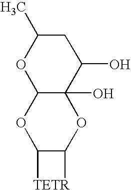

piperidines, such as piperidines anellated with another ring, e.g. quinolizines, such as octahydroquinolizines, e.g. of formula

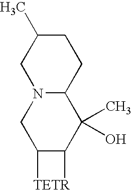

wherein TETR together with the two carbon atoms to which TETR is attached is a tetracyclic compound of formula TETR as defined above.

Each of the rings A, B, D and E comprises unsubstituted rings and substituted rings, e.g. unsubstitute or one or more fold substituted. Anellated rings may be substituted or unsubstituted.

Ring substituents include appropriate substituents, e.g. such as conventional in steroids, e.g. including one or more
halogen, such as fluoro, chloro, bromo,
amino, including unsubstituted amino and substituted amino, such as amino, di(C₁₋₆)alkylamino, e.g. including di(C₁₋₆)alkylamino-di(C₁₋₆)alkylamino,
hydroxyimino,
hydroxy,
a keto group,
formyl,
(C₁₋₁₂)alkyl, including unsubstituted alkyl, and substituted alkyl, e.g. alkyl substituted by
   sulfonyl,
   amino, e.g. including (di(C₁₋₄)alkylamino, amino(C₁₋₆)alkylamino(C₁₋₆)alkylamino,
   hydroxy,
   (C₂₋₆)alkenylcarbonyl, e.g. including (C₁₋₄)alkylcarbonyloxy(C₂₋₆)alkenylcarbonyl,
   cyano,
   heterocyclyl having 5 to 6 ring members and one to 4 heteroatoms selected from N, O and S, such as dihydrofurane, dihydropyrane, piperidinyl, e.g. substituted by (C₁₋₄)alkyl, a keto group,
   (C₁₋₄)alkylcarbonyloxy, e.g. including hydroxycarbonyl(C₁₋₄)alkylcarbonyloxy,
   heterocyclylcarbonyloxy, wherein heterocyclyl has 5 or 6 ring members and 1 to 4 heteroatoms selected from N, O and S, including unsubstituted heterocyclyl and substituted heterocyclyl, e.g. substituted one or morefold by (C₁₋₄)alkyl,
   (C₁₋₆)alkylaminocarbonyl, e.g. including hydroxycarbonyl(C₁₋₄)alkylaminocarbonyl. sulfonyl(C₁₋₄)alkyl-di(C₁₋₄)alkylammonium(C₁₋₄)alkylaminocarbonyl,
   (C₆₋₁₈)arylaminocarbonyl, such as phenylaminocarbonyl, e.g. including di-trifluoromethylphenyl-aminocarbonyl,
   hydroxycarbonyl,
halo(C₁₋₄)alkyl, such as trifluoromethyl,
a group ═CH₂, or ═CH—OH,
(C₂₋₁₂)alkenyl, e.g. including unsubstituted and substituted (C₂₋₁₂)alkenyl, e.g. substituted by hydroxycarbonyl,
(C₂₋₆)alkynyl,
(C₆₋₁₈)aryl, such as phenyl, e.g. di(C₁₋₄)alkylaminophenyl,
(C₁₋₄)alkoxy, e.g. including unsubstituted alkoxy and substituted alkoxy, e.g. alkoxy substituted by
   hydroxy, halogen, (C₁₋₄)haloalkyl, such as CCl₃, e.g. see cloxotestosterone,
(C₁₋₄)alkylthio,
(C₃₋₈)cycloalkyloxy, e.g. cyclopentyloxy,
(C₃₋₈)cycloalkenyloxy, e.g. cyclopentenyloxy,
hydroxycarbonyl,
(C₁₋₆)alkoxycarbonyl,
(C₁₋₄)alkylthiocarbonyl,
(C₁₋₄)alkoxycarbonyloxy,
(C₂₋₄)alkenyloxycarbonyloxy, e.g. including phenyl(C₂₋₄)alkenyloxycarbonyloxy, e.g. wherein phenyl is substituted by hydroxy and (C₁₋₄)alkoxy,
(C₁₋₄)alkylthiocarbonyl, e.g. including fluoro(C₁₋₄)alkylthiocarbonyl,
(C₁₋₄)alkylcarbonyl, e.g. including halo(C₁₋₄)alkylcarbonyl, hydroxy(C₁₋₄)alkylcarbonyl, (C₁₋₄)alkylcarbonyl(C₁₋₄)alkylcarbonyl, (C₁₋₄)alkylcarbonyloxy(C₁₋₄)alkylcarbonyl, di(C₁₋₄)alkylamino(C₁₋₄)alkylcarbonyloxy(C₁₋₄)alkylcarbonyl, hydroxycarbonyl(C₁₋₄)alkylcarbonyloxy(C₁₋₄)alkylcarbonyl, mercapto(C₁₋₄)alkylcarbonyl, heterocyclyl(C₁₋₄)alkylcarbonyl, wherein heterocyclyl has 1 to 4 heteroatoms selected from N, O, S and 5 to 6 ring members, e.g. piperazinyl,
(C₁₋₈)alkylcarbonyloxy,
(C₁₋₄)alkylcarbonylthio,
(C₂₋₈)alkenylcarbonyloxy,
heterocyclylcarbonyloxy, wherein heterocyclyl has 1 to 4 heteroatoms selected from N, O, 5 and 5 to 6 ring members, e.g. including furane,
aminocarbonyloxy, e.g. including di(C₁₋₄)alkylaminocarbonyloxy, such as dichloroethylaminocarbonyloxy,
cyano,
heterocyclyl having 5 or 6 ring members and 1 to 4 heteroatoms selected from N, O and S, including unsubstituted heterocyclyl and substituted heterocyclyl, e.g. substituted by (C₁₋₄)alkyl, hydroxy or keto groups, e.g. piperidinyl, morpholino,
heterocyclyloxy, wherein heterocyclyl has 5 or 6 ring members and 1 to 4 heteroatoms selected from N, C and S, including unsubstituted heterocyclyl and substituted heterocyclyl, e.g. substituted by hydroxy, (C₁₋₄)alkyl, or (C₁₋₄)alkoxy, e.g. including a sugar residue, bound to a compound of formula TETR via an oxygen atom, which sugar residue itself may be further substituted one or morefold via an oxygen atom by further sugar residues,

Substituents in the meaning of substituted heterocyclyl and substituents in rings anellated to a ring in a compound of formula TETR include such as described above for a compound of formula TETR.

Compounds of formula TETR are steroids. Steroids include natural and chemically modified corticoids, such as pharmaceutically active corticoids and derivatives thereof, e.g. including corticosteroids, such as glucocorticoids (i.e. having glucocorticoid-like activity), e.g. which show pharmaceutical activity,

e.g. including steroids in free form and in the form of
esters, e.g. including mono- and diesters, e.g. in the form of salts, e.g. sodium,
acetals and ketals, such as acetonides,
   e.g. in the form of salts and solvates, where applicable.

Examples of compounds of formula TETR include such as described in The Merck Index, 12th and 13th edition, e.g. including 21-acetoxypregnenolone, adonitoxin, adrenosterone, alclomethasone, (e.g. diproprionate), aldosterone, alfadolone acetate, alfaxalone, algestone (e.g. acetophenide), allocholesterol, allopregnane (e.g. and -ols and ones thereof), allotetrahydrocortisone, allylesterenol, altrenogest, amcinonide, anagestone, androisoxazole, androstane (e.g. ols and ones), androstenediol (e.g. acetate, diacetate, benzoate, diproprionate), androstenedione, androst-16-en-3-ol, androsterone, antheridiol, alphaantiarin, azacosterol, batrachotoxin, beclomethasone (fludroxycortide) (e.g. -dipropionate), betamethasone (e.g. -acetate, -benzoate, -dipropionate, sodium phosphate, -valerate), bolandiol, bolasterone, boldenone, budesonide, bufalin, bufogenin B, bufotalin, bufotoxin, calotropin, calusterone, campesterol, canrenone, cephalosporin P₁, CHAPS, chenodiol, chlormadinone (e.g. acetate), chlorogenin, chinoprednisone, cholane, cholanic acid, 5alpha-cholestane, cholestanol, cholesterol, cholic acid, chondrillasterol, ciclesonide, cimigenol, cinobufotalin, clobetasol (e.g. propionate), clobetasone (e.g. butyrate), clocortolone (e.g. -acetate, -pivalate), clomestrone, cloprednol, clostebol, cloxotestosterone, colpornon, conessine, convallamarogenine, convallatoxin, coproergostane, coprostane, coprosterol, corticosterone, cortisol (hydrocortisone), cortisone (e.g. -acetate), cortivazol, cortol, cortolone, cucurbitacins (e.g. A, B, C, D, F, P, R), cyclobuxine D, cynanchogenin, cyproterone, danazol, deflazacort, 3-beta-7-dehydrocholesterol, dehydrocholic acid, 11-dehydrocorticosterone, dehydroergosterol, 7-dehydrositosterol, delmadinone (e.g. acetate), demegestone, deoxycholic acid, deoxycorticosterone (e.g. -acetate), descinolone (e.g. -acetonide), deslanoside, desmosterol, desogestrol, desonide, desoximetasone, 11-desoxy-17-hydroxycorticosterone, dexamethasone (e.g. sodium phosphate, -acetate, -isomicotinate), dichiorisone, dienogest, diflorasone (e.g. diacetate), difluocortolone (e.g. -pivalate, valerate), difluprednate, digalogenin, diginatigenin, diginin, digitalin, digitogenin, digitoxigenin, digoxigenin, dihydroequiline, dimethisterone, dinosterol, diosgenin, dromostanolone (masterone), drospirenone, dutasteride, dydrogesterone, ecdysteroids (ecdysone, 20-hydroxyecdysone), epiandrosterone, epicholestanol, epicholesterol, 16-epiestriol, epinestrol, epitiostanol, epostane, epristeride, equilenin, equilin, ergostane, ergostanol, alpha-, beta- and gamma-ergostenol, ergosterol, (alpha-)estradiol, estramustine, estriol, estrone, ethinyl estradiol, ethisterone, ethylestrenol, ethynodiol, etiocholane, etiocholanic acid, etonogestrel, exemestane, finasteride, fluazacort, flucloronide, fludrocortisone, flumedroxone acetate, flumethasone (e.g. -pivalate), flunisolide, fluocinolone (e.g. -acetonide), fluocinonide, fluocortin butyl, fluocortolone (e.g. -caproate), fluorometholone, fluoxymesterone (halotestin), fluperolone (e.g. -acetate), fluprednidene (e.g. -21-acetal, -acetate), fluprednisolone (e.g. -valerate), flurandrenolide, fluorogestone (e.g. acetate), fluticasone (e.g. -propionate, valerate), formebolone, formestane, formocortal, fucosterol, fungisterol, funtumine, furazabol, fusidic acid, fusidic acid, gamabufotaline, ganaxolone, gentrogenin, gestodene, gestonorone (e.g. caproate), gestrinone (including tetrahydrogestrinone), gitogenin, F-gitonin, gitoxigenin, gitoxin, alpha- and beta-acetylgitoxin, glycocholic acid, gratiogenin, halcinonide, halobetasol (e.g. -propionate), halomethasone (e.g. monohydrate), halopredone (e.g. acetate), hecogenin, hellebrin, helvolic acid, holarrhenine, hydrallostane, hydrocortamate, hydrocortisone (e.g. -acetate, -buteprat, -butyrate, cypionate, -sodium phosphate, -sodium succinate, -hemisuccinate, -valerate), 24- and 25-hydroxycholesterol, hydroxydione (sodium), 17-hydroxy-16-methylene-Δ⁶-progesterone, 17alpha-hydroxyprogesterone, hyodeoxycholic acid, isoestradiol, 8-isoestrone, isoflupredone, isopyrocalciferol, isorubijervine, 11-ketoprogesterone, kurchessine, kurcholessine, lanosterol, lithocholic acid, loteprednol (e.g. etabonate), lumisterol, lynestrenol, maziprednone, medrogestone, medroxyprogesterone, medrysone, megestrol (acetate), melengestrol, mepitiostane, meprednisone, mestanolone, mesterolone, mestranol, methandriol, methandrostenolone (dianabol), methenolone, methylprednisolone (e.g. -acetate, -sodium phosphate, -sodium succinate, aceponate), 17-methyltestosterone, 17alpha-methyltestosterone 3-cyclopentyl enol ether, methyltrienolone, mibolerone, mometasone (e.g. fuorate), moxestrol, mytatrienediol, nandrolone, neoergosterol, neriifolin, nivazol, norbolethone, norcholanic acid, norethandrolone, norethindrone, norethynodrel, norgesterone, norgestimate, norgestrel, norgestrienone, normethandrone, norvinisterone, oleandrin, onapristone, gamma-oryzanol, osaterone, ouabagenin, ouabain, oxabolone, oxandrolone, oxendolone, oxymesterone, oxymetholone, pancuronium halogenide (bromide), paramethasone (e.g. -acetate), pavoninin-5, pentagestrone, periplogenin, peruvoside, phenesterine, pipecuriom halogenide (bromide), ponasterone A, prasterone, prednicarbate, prednimustine, prednisolone (e.g. including -acetate, -hemisuccinate, -sodium phosphate, -sodium succinate, -tebutate, 21-diethylaminoacetate), prednisone, prednival, prednylidene, pregnane, pregnanediol, 3,20-pregnanedione, pregnan-3alpha-ol-20-one, 4-pregnene-20,21-diol-3,11-dione, 4-pregnene-11beta,17alpha,20beta,21-tetrol-3-one, 4-pregnene-17alpha,20beta,21-triol-3,11-dione, 4-pregnene-17alpha,20beta,21-triol-3-one, pregnenolone, progesterone, promegestone, proscillaridin, pyrocalciferol, quinbolone, quinestradiol, quinestrol, rapacuronium halogenide (bromide), resibufogenin, rimexolone, rocuronium halogenide (bromide), rofleponide (e.g. palmitate), rubijervine, samanderine, sarmentogenin, sarsasapogenin, sarverogenin, scillaren (A and B), scillarenin, scilliroside, alpha-,beta- and gamma-sitosterol, solanidine, solanocapsine, solasodine, alpha-spinasterol, spironolactone, squalamine halogenide (trihydrochloride), stanolone, stanozolol, stenbolone, stigmastanol, stigmasterol, stophanthidin, tanghinigenin, taurocholic acid, testosterone, tetrahydrocortisone, thevetin A, tibolone, ticabesone (e.g. -propionate), tigogenin, tiomesterone, tipredane, tirilazad, tixocortol, tomatidine, tralonide, trenbolone, trengestone, triamcinolone (e.g. -acetonide, -acetonide sodium phosphate, -diacetate, benetonide, hexacetonide), trilostane, ursodiol, uscharidin, uzarigenin, uzarin, vercuronium halogenide (bromide), veralkamine, withaferin A, e.g., and pharmacodynamic equivalents thereof, preferably aclomethasone, beclomethasone, betamethasone, clobetasole, hydrocortisone, dexamethasone, difluocortolene, fluticasone, halobetasol, halomethasone and mometasone, such as e.g. hydrocortisone, betamethasone, e.g. betamethasone 17-valerate, dexamethasone, mometasone, e.g. mometasone furoate, or clobetasol, e.g. clobetasol-17-butyrate, more preferably hydrocortisone, mometasone, clobetasol and triamcinolone.

In another aspect the present invention provides a tetracyclic compound
for use, or
in a method
   according to the present invention wherein a tetracyclic compound is a steroid, such as a compound of formula TETR, e.g. a corticoid, such as a corticoid, e.g. selected from the group consisting of hydrocortisone, betamethasone, e.g. betamethasone 17-valerate, dexamethasone, mometasone, e.g. mometasone furoate, and clobetasol, e.g. clobetasol-17-butyrate, more preferably hydrocortisone, mometasone, triamcinolone and clobetasol.

Pharmacodynamic equivalents are meant to include corticoids, showing pharmaceutical activity similar with specific corticoids listed herein.

A method according to the present invention may be used for improved treatment of disorders, such as diseases, e.g. including skin disorders, e.g. wherein the disorder is a disorder in which T lymphocytes (T cells) are involved in the pathophysiology of the disorder, such as T-cell mediated acute or chronic inflammatory disorders or autoimmune disorders.

In another aspect the present invention provides a method of treatment, e.g. topical (epicutaneous) treatment, of disorders in which T cells (i.e. T lymphocytes) are involved in the pathophysiology of the disorder, comprising administering, e.g. topically (epicutanously), to a subject in need of such treatment an effective amount of a tetracyclic compound, and administering, e.g. simultaneously or in consequence, e.g. in consequence, an effective amount of a T-cell modulator, preferably a calcineurin inhibitor, more preferably a compound of formula I, II or III.

The principle of topical (epicutanous) treatment with a tetracyclic compound and a pharmaceutically active compound according to the present invention may be applied e.g. for
anti-inflammatory, e.g. including acne, treatment,
treatment of hyperproliferative disorders,
treatment of phototoxic conditions (solar dermatoses),
anti aging treatment,
topical analgetic treatment,
anti-hairloss treatment,
antiperspirant treatment,
anti-pruritic treatment, and
astringent agent.

Treatment includes prophylaxis, prevention, medication and/or therapy.

In another aspect the present invention provides a method for topical (epicutanous) treatment of
inflammatory disorders, e.g. including acne,
microbial disorders,
hyperproliferative disorders,
phototoxic conditions, e.g. including solar dermatoses, hairloss, or
conditions wherein an analgetic, anti-aging, antiperspirant, anti-pruritic agent or astringent agent is helpful,
   comprising topically (epicutaneously) administering an effective amount of
a tetracyclic compound, and
a pharmaceutically active compound selected from the group consisting of
   an anti-inflammatory agent, e.g. including an anti-acne agent,
   antimicrobial,
   anti-hyperproliferative agent,
   anti-phototoxic agent,
   anti-hairloss agent,
   analgetic agent,
   anti-aging agent,
   antiperspirant, and
   anti-pruritic agent
      to a subject in need of such treatment;
      e.g. wherein a tetracyclic compound is administered before or simultaneously, preferably before the pharmaceutically active compound.

Inflammatory disorders, e.g. including skin disorders, which may be treated by topical administration by a method of the present invention, e.g. include psoriasis, atopic eczema, seborrheic dermatitis, intertrigo, nummular eczema, irritant or allergic contact dermatitis, urticaria, parapsoriasis, lichen simplex chronicus, acute/chronic dyshidrotic eczema, lupus erythematosus, pemphigus, lichen planus, granuloma annulare, acne, alopecia greata, cutaneous Graft vs Host reactions, vasculitides or pyoderma gangrenosum; preferably psoriasis, atopic eczema, seborrheic dermatitis, irritant or allergic contact dermatitis, lichen planus, lichen rubber, alopecia greata, pyoderma gangrenosum. Inflammatory disorders which may be treated by a method according to the present invention include such which are mediated by a T-cell modulator, e.g. a calcineurin inhibitor.

In another aspect the present invention provides a method for topical (epicutanous) treatment, of inflammatory disorders, e.g. skin disorders, selected from the group consisting of psoriasis, atopic eczema, seborrheic dermatitis, intertrigo, nummular eczema, irritant or allergic contact dermatitis, urticaria, parapsoriasis, lichen simplex chronicus, acute/chronic dyshidrotic eczema, lupus erythematosus, pemphigus, lichen planus, granuloma annulare, acne, alopecia greata, cutaneous Graft vs Host reactions, vasculitides, and pyoderma gangrenosum, such as psoriasis, atopic eczema, seborrheic dermatitis, irritant or allergic contact dermatitis, lichen planus, lichen rubber, alopecia greata, pyoderma gangrenosum, comprising topically (epicutaneously) administering an effective amount of a tetracyclic compound, and an effective amount of a topically (epicutaneously) effective, anti-inflammatory agent,

e.g. a T-cell modulator,
such as a calcineurin inhibitor,
e.g. a compound of formula I, II or III, preferably
a compound of formula I_{P} (pimecrolimus),
a compound of formula II_{FK} (tacrolimus),
a compound of formula III wherein R is ethyl (cyclosporin A),
   more preferably a compound of formula I or of formula II;
   to a subject in need of such treatment.

Hyperproliferative disorders, e.g. including skin disorders, which may be treated by topical administration by a method of the present invention, e.g. include psoriasis, T cell lymphoma, pseudolymphoma, actinic keratosis, warts, precancerous lesions, benign epithelial tumors, keratoacanthomas, squamous cell carcinoma, basel cell carcinoma.

Hyperproliferative disorders which may be treated by a method according to the present invention include such which are mediated by a T-cell modulator, e.g. a calcineurin inhibitor.

In another aspect the present invention provides a method for topical (epicutanous) treatment of hyperproliferative disorders, e.g. skin disorders, selected from the group consisting of psoriasis, actinic keratosis, warts, precancerous lesions, benign epithelial tumors, keratoacanthomas, squamous cell carcinoma, basel cell carcinoma, such as actinic keratosis, warts, precancerous lesions, benign epithelial tumors, keratoacanthomas, squamous cell carcinoma, basel cell carcinoma, comprising topically (epicutaneously) administering an effective amount of a tetracyclic compound, and an effective amount of a topically (epicutaneously) effective anti-hyperproliferative agent,

e.g. a T-cell modulator,
such as a calcineurin inhibitor,
e.g. a compound of formula I, II or III, preferably
a compound of formula I_{P} (pimecrolimus),
a compound of formula II_{FK} (tacrolimus),
a compound of formula III wherein R is ethyl (cyclosporin A),
   more preferably a compound of formula I or of formula II;
   to a subject in need of such treatment.

In another aspect the present invention provides a method for the preparation of a medicament for the treatment of disorders, e.g. for topical (epicutaneous treatment, in which T lymphocytes are involved in the pathophysiology of the disorder, comprising combining an effective amount of a tetracyclic compound and an effective amount of a T-cell modulator, preferably a calcineurin inhibitor, more preferably a compound of formula I, II or III, e.g. together with an indication for administering topically (epicutanously), an effective amount of said agent, e.g. simultaneously or subsequently, preferably subsequently to a tetracyclic compound.

In another aspect the present invention provides a method for the preparation of a medicament for treating topically (epicutanously) inflammatory disorders, e.g. including acne, microbial disorders, hyperproliferative disorders, phototoxic conditions (solar dermatoses), hairloss, or conditions wherein an analgetic, anti-aging, antiperspirant, anti-pruritic agent or astringent agent is helpful, comprising combining an effective amount of
a tetracyclic compound, and
a pharmaceutically active compound selected from the group consisting of an anti-inflammatory agent, e.g. including an anti-acne agent, antimicrobial, anti-hyperproliferative agent, anti-phototoxic agent, anti-hairloss agent, analgetic, anti-aging agent, antiperspirant, and anti-pruritic and astringent,
   e.g. together with an indication for administering topically (epicutanously), an effective amount of said agent, e.g. simultaneously or subsequently, preferably subsequently to a tetracyclic compound.

In another aspect the present invention provides a method for the preparation of a medicament for topical (epicutaneous) treatment of inflammatory disorders, e.g. skin disorders, selected from the group consisting of psoriasis, atopic eczema, seborrheic dermatitis, intertrigo, nummular eczema, irritant or allergic contact dermatitis, urticaria, parapsoriasis, lichen simplex chronicus, acute/chronic dyshidrotic eczema, lupus erythematosus, pemphigus, lichen planus, granuloma annulare, acne, alopecia greata, cutaneous Graft vs Host reactions, and pyoderma gangrenosum,

such as psoriasis, atopic eczema, seborrheic dermatitis, irritant or allergic contact dermatitis, lichen planus, alopecia greata, pyoderma gangrenosum,
comprising combining an effective amount of a tetracyclic compound, and an effective amount of an antiinflammatory agent, e.g. a T-cell modulator, such as a calcineurin inhibitor,
e.g. a compound of formula I, II or III,
e.g. together with an indication for administering topically (epicutanously), an effective amount of said agent, e.g. simultaneously or subsequently, preferably subsequently to a tetracyclic compound.

In another aspect the present invention provides a method for the preparation of a medicament for topical (epicutaneous) treatment of hyperproliferative disorders, e.g. skin disorders, selected from the group consisting of psoriasis, actinic keratosis, warts, precancerous lesions, benign epithelial tumors, keratoacanthomas, squamous cell carcinoma, basel cell carcinoma;

such as actinic keratosis, warts, precancerous lesions, benign epithelial tumors, keratoacanthomas, squamous cell carcinoma, basel cell carcinoma,
comprising combining an effective amount of a tetracyclic compound, and an effective amount of an antihyperpoliferative agent, e.g. a T-cell-modulator, such as a calcineurin inhibitor, e.g. a compound of formula I, II or III,
e.g. together with an indication for administering topically (epicutanously), an effective amount of said agent, e.g. simultaneously or subsequently, preferably subsequently to a tetracyclic compound.

In a method for treating disorders according to the present invention a tetracyclic compound is administered before or simultaneously, preferably before the pharmaceutically active compound, e.g. the pharmaceutically active compound is administered subsequently to a tetracyclic compound, e.g. the disorder is topically (epicutanously) pre-treated with a tetracyclic compound and subsequently topically (epicutanously) treated with a pharmaceutically active compound.

A tetracyclic compound and/or a pharmaceutically active compound for use or in a method according to the present invention independently of each other may be in free form, in the form of a salt, in solvate form or in the form of a salt and a solvate, where salts and/or solvates exist.

In another aspect the present invention provides a tetracyclic compound
for use, or
in a method
   according to the present invention, wherein a pharmaceutically active compound is in the form of a salt.

In another aspect the present invention provides a tetracyclic compound in the form of a salt;
for use, or
in a method
   according to the present invention, wherein a pharmaceutically active compound is in free form.

In another aspect the present invention provides a tetracyclic compound in the form of a salt;
for use, or
in a method
   according to the present invention, wherein a pharmaceutically active compound is in the form of a salt.

Treatment includes treatment, e.g. therapy, and prevention, e.g. prophylaxis. For such treatment, the appropriate dosage will, of course, vary depending upon, for example, the chemical nature and the pharmacokinetic data of a compound of the present invention employed, the individual host, the mode of administration and the nature and severity of the conditions being treated. However, in general, for satisfactory results in larger mammals for example humans, a pharmaceutically active compound may be used in similar dosage ranges as conventionally used in therapies, e.g. in dosage ranges as known for a therapy with such pharmaceutically active compound, e.g. a calcineurin inhibitor, e.g. pimecrolimus or tacrolimus, may be provided as a solution or cream (gel) in the range from about 0.1% to 5% w/v or w/w when administered locally, wherein the dosage will depend on the kind and severity of the disease to be treated e.g. a tetracyclic compound, such as a steroid, e.g. a corticoid, is given in dosages as known for standard therapies, such as e.g. in a range of 0.5 to 5% in case of topical application (or in a range of 0.25 to 2500 mg, preferably 1 to 500 mg, such as 1 to 50 mg, when administered systemically, e.g. orally). The ratio of a pharmaceutically active compound to a tetracyclic compound according to the present invention is not critical. A tetracyclic compound is used in an amount which is effective to enhance penetration/permeability of the (inflamed) skin by a pharmaceutically active compound, and a pharmaceutically active compound is used in an amount effective for treating a corresponding disease for which the pharmaceutically active compound is useful. According to a method of the present invention the amount of the pharmaceutically active compound may be even lower compared with administration to untreated skin, e.g. because of better and deeper skin permeation/penetration.

According to the use and in a method of the present invention
one or more tetracyclic compounds may be used, preferably one single tetracyclic compound is used;
one or more pharmaceutically active compounds may be used, preferably one single pharmaceutically active compound is used.

A tetracyclic compound and a pharmaceutically active compound of the present invention may be used, e.g. administered, in free form or in the form of a pharmaceutically acceptable salt, e.g. an acid addition salt or metal salt; optionally in the form of a solvate. Tetracyclic compounds may additionally be in the form of esters, acetonides, e.g. and additionally in the form of salts. The tetracyclic and pharmaceutically active compounds of the present invention in the form of a salt/ester/acetonide/solvate/exhibit the same order of activity as the compounds used in the present invention in free form; optionally in the form of a solvate. Pharmaceutically active compounds of the present invention, such as T-cell modulators, e.g. calcineurin inhibitors, e.g. including compounds of formulae I, II and III, and tetracyclic compounds according to the present invention are known or may be obtained according, e.g. analogously, to a method as conventional.

In the following examples temperatures are given in degrees Celsius (° C.) and are uncorrected.

ASD732 used as a reference compound is a compound of formula

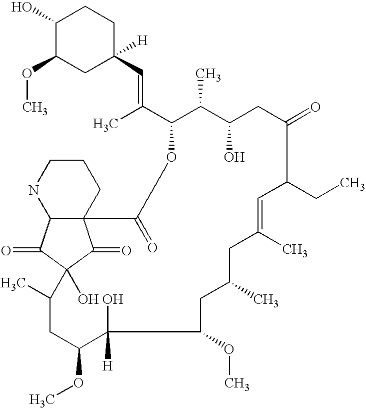

The following abbreviations are used:
CS corticosteroids
CLO clobetasol-17-propionate
ESI electrospray ion source
HC hydrocortisone
HPLC high pressure liquid chromatography
MOM mometasone furoate
MS mass spectroscopy
PBS phosphate buffered saline
RP reverse phase
RPLC reverse phase liquid chromatography

### Methods

### EXAMPLE A

### Pre-Treatment of Porcine Skin In Vivo, Collection of Skin Samples

Three young, male castrated domestic pigs with body weights of approximately 15 kg are used as skin donors. The animals are obtained from a local breeder and maintained under standardized conditions (22±1°, 55±5% relative humidity, 10 changes of fresh air/hour, 12 hours day/12 hours night cycle). After a settling-in period of 7 days the pigs are treated topically on test areas on the dorsolateral back
with CS solutions in EtOH/propylene glycol (3+7, v/v) as a solvent, and
contra-laterally with solvent alone,
   twice daily for 5 days.

HC (Sigma) at 1.0%, MOM (Sequoia Research Products) at 0.1% and CLO (Sigma) at 0.05% are used as CS. The CS-samples and solvent alone are applied in volumes of 50 μl to skin areas of 3.5×6.5 cm each. All three CS and solvent alone are applied to all animals. 16 hours after the last application the animals are killed and the treated skin samples are dissected. Full-thickness skin samples are dissected from vehicle- and CS-treated sites and cut into 0.4 mm split-thickness skin samples with a dermatome (Aesculap®). The samples include the epidermal layers and the superficial part of the dermis but no deep dermal layers and no subcutaneous fat. Tissue from the same animals are used for testing penetration and permeation rate of 2 different pharmaceutically active test compounds, namely a compound of formula I_{P} (pimecrolimus) and a compound of formula II_{FK} (tacrolimus), in the skin penetration/permeation assay, see Example B below.

### EXAMPLE B

### Skin Penetration/Permeation Assay

Percutaneous penetration is studied in static Franz-type diffusion cells with 2.54 cm² exposed skin areas and receptor chamber volumes from 5.30 to 6.05 ml, see e.g. Schmook F B, Stütz A, Reinhardt J, Skin Pharmacol 1993; 6: 116-124. PBS/EtOH 3:1 is used as receptor phase. All experiments are performed at 32° in triplicates for 48 hours. Test compounds are applied to the epidermal side of the skin samples in their marketed formulations, i.e. Elidel® 1% cream (pimecrolimus) and Protopic® 0.1% ointment (tacrolimus), each in amounts of 300 mg. Samples of 100 μl are taken from the receptor phase at 5 time points during the 48-hours experiment and replaced by fresh receptor fluid. After addition of an internal standard (ASD732, see e.g. EP569337) the samples are directly analyzed by RPLC using MS-based detection. After termination of the experiment, the skin is removed from the diffusion cells and the stratum corneum is peeled off with 20 strippings with a transparent adhesive tape (Kores®). Specimens from the stripped skin are taken with a biopsy punch, weighed and then homogenized in 0.1 M sodium phosphate buffer, pH 7. Following addition of an internal standard (ASD732) and buffer of pH 10 to the homogenates, extraction is performed with tert-butyl methylether. Solvent from each extract obtained is evaporated, each residue obtained is redissolved and subjected to analysis by RPLC using MS-based detection.

### EXAMPLE C

### Analytical Methods for Receptor Fluid and Skin Samples

Receptor fluid samples are analyzed by RP-HPLC with MS detection in ESI positive mode (LC-MS/MS analysis). The MS/MS mode is used to increase selectivity. LC-MS/MS analysis is carried out with an Agilent 1100 Series Capillary LC System coupled to a Finnigan LCQ mass spectrometer. A Phenomenex Luna C18(2) column (3 μm, 100×2 mm) equipped with a precolumn is used and eluted isocratically with a flow rate of 100 μl/minute at 60°. The effluent is delivered unsplit to the ESI ion-source. Under the chromatographic conditions used, all compounds tested yield a strong sodium adduct. For MS/MS parent ions are selected. The quantification of the parent ions is based on the area ratio of the fragment ions to the fragment ion of the internal standard.

### EXAMPLE D

### Penetration/Permeation Profiles

### Results

The concentrations of pimecrolimus and tacrolimus in the various skin samples (indicating skin penetration) as well as the data on the permeation rate (a measure of compound passing the skin barrier) are summarized in TABLE 1 below.

TABLE 1

| Pre-Treatment | Formulation | Skin concentration | Permeation rate |
|---|---|---|---|
| None | Elidel cream, 1% | 2.49 ± 0.36 | 0.13 ± 0.04 |
| HC | Elidel cream, 1% | 3.95 ± 0.76 | 0.49 ± 0.05 |
| MOM | Elidel cream, 1% | 4.33 ± 1.18 | 0.38 ± 0.00 |
| CLO | Elidel cream, 1% | 3.46 ± 0.31 | 0.53 ± 0.05 |
| None | Protopic 0.1% | 3.55 ± 0.34 | 1.45 ± 0.53 |
| HC | Protopic 0.1% | 6.47 ± 1.76 | 2.87 ± 0.17 |
| MOM | Protopic 0.1% | 4.31 ± 0.95 | 2.70 ± 0.56 |
| CLO | Protopic 0.1% | 3.02 ± 0.26 | 1.86 ± 0.53 |

In TABLE I
Skin concentration is indicated in μg/g
Permeation rate is indicated in ng/ml/hour.

From TABLE 1 it is evident that
the concentrations of active compounds in untreated and pre-treated skin samples are in a comparable range, despite of the different drug concentration in Elidel cream 1% and Protopic ointment 0.1%
in corticosteroid pretreated samples permeation rates of pimecrolimus are increased by factors 3.8 (HC-pre-treatment), 2.9 (MOM pre-treatment) and 4.1 (CLO pre-treatment) compared with untreated skin
the differences are less pronounced with tacrolimus (factors 1.3-1.9)
the permeation rate of tacrolimus in comparison with pimecrolimus, is 11.2 times higher in untreated skin, 5.9 times higher in HC-pre-treated skin, 7.1 times higher in MOM-pre-treated skin and 3.5 times higher in CLO-pretreated skin
the skin penetration (skin concentration) is enhanced for pimecrolimus (at least about 10%) but not significantly for tacrolimus, in pre-treated skin compared with untreated skin.

1. A tetracyclic compound for use in the enhancement of skin permeability.

2. A tetracyclic compound for use in the enhancement of skin permeability for a pharmaceutically active compound, which pharmaceutically active compound is other than a tetracyclic compound according to the present invention.

3. A tetracyclic compound for use according to claim 1, wherein the use is a topical use.

4. A method of enhancing the skin permeability for a pharmaceutically active compound comprising treating the skin with a tetracyclic compound and administering a pharmaceutically active compound.

5. The method of claim 4, wherein the tetracyclic compound and the pharmaceutically active compound both are administered topically.

6. A method for treatment of disorders in which T cells are involved in the pathophysiology of the disease comprising administering to a subject in need of such treatment an effective amount of a tetracyclic compound and an effective amount of a T-cell modulator.

7. A method for the preparation of a medicament for the treatment of disorders in which T cells are involved in the pathophysiology of the disorder comprising combining an effective amount of a tetracyclic compound and an effective amount of a T-cell modulator.

8. A method according to claim 6, wherein the treatment is topical treatment.

9. A method according to claim 8, wherein disorders are inflammatory disorders selected from the group consisting of psoriasis, atopic eczema, seborrheic dermatitis, intertrigo, nummular eczema, irritant or allergic contact dermatitis, urticaria, parapsoriasis, lichen simplex chronicus, acute/chronic dyshidrotic eczema, lupus erythematosus, pemphigus, lichen planus, lichen rubber, granuloma annulare, acne, alopecia greata, cutaneous Graft vs Host reactions, vasculitides and pyoderma gangrenosum and wherein a T-cell-modulator is an antiinflammatory agent.

10. A method according to claim 8, wherein disorders are hyperproliferative disorders selected from the group consisting of psoriasis, T cell lymphoma, pseudolymphoma, actinic keratosis, warts, precancerous lesions, benign epithelial tumors, keratoacanthomas, squamous cell carcinoma, basel cell carcinoma, comprising topically and wherein a T-cell-modulator is an antihyperproliferative agent.

11. A method according to claim 6, wherein a T-cell modulator, an antiinflammatory agent, or an antihyperproliferative agent is a calcineurin inhibitor.

12. A method according to claim 11, wherein a calcineurin inhibitor is a compound of formula 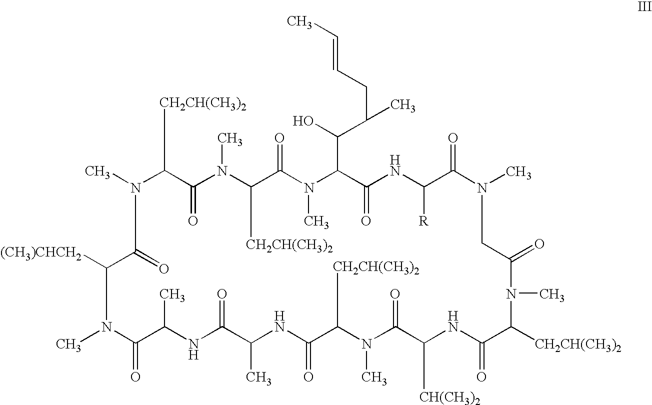
wherein R is methyl, ethyl, propyl, isopropyl or —CH(OH)CH₃, preferably R is ethyl (Cyclosporin A).

13. A method according to claim 11, wherein a calcineurin inhibitor is a compound of formula 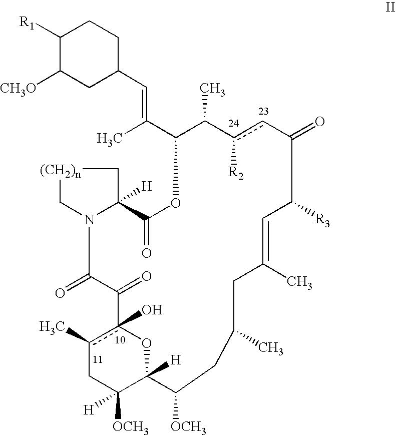
wherein
R₁ is hydroxy or protected hydroxy,
R₂ is hydrogen, hydroxyl or protected hydroxyl,
R₃ is methyl, ethyl, propyl or allyl,
n is an integer of 1 or 2, and the dotted line is a bond, or is no bond, preferably a compound of formula 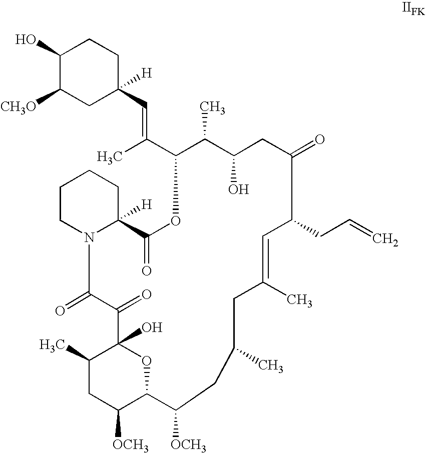
(tacrolimus, FK506).

14. A method according to claim 11, wherein a calcineurin inhibitor is a compound of formula 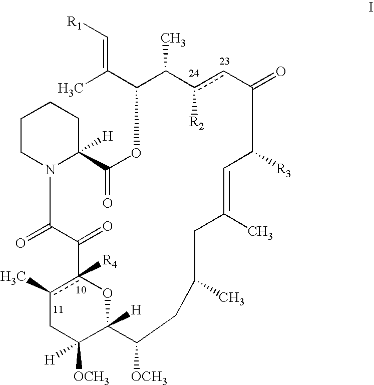
wherein either
R₁ is a group (a) of formula 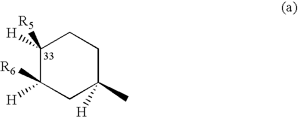
wherein
R₅ is chloro, bromo, iodo or azido,
R₆ is hydroxy or methoxy, and
R₄ is hydroxy and there is a single bond in 10,11 position; or absent, and there is a double bond in 10,11 position,
or
R₁ is a group (b) or (c) of formula 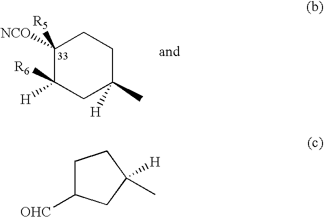
wherein
R₆ is as defined above, and
R₄ is hydroxy and there is a single bond in 10,11 position,
R₂ is oxo and there is a single bond in 23,24 position; hydroxy and there is a single or double bond in 23,24 position; or absent and there is a double bond in 23,24 position; and
R₃ is methyl, ethyl, propyl or allyl.

15. A method of topical treatment of inflammatory disorders, acne, microbial disorders, hyperproliferative disorders, phototoxic conditions (solar dermatoses), hairloss, or conditions wherein an analgetic, anti-aging, antiperspirant, anti-pruritic or astringent is helpful, comprising topically administering an effective amount of a tetracyclic compound, and a pharmaceutically active compound selected from the group consisting of an anti-inflammatory agent, anti-acne agent, antimicrobial, anti-hyperproliferative agent, anti-phototoxic agent, anti-hairloss agent, analgetic, anti-aging agent, antiperspirant, anti-pruritic and astringent to a subject in need of such treatment.

16. A method for the preparation of a medicament for treating topically inflammatory disorders, acne, microbial disorders, hyperproliferative disorders, phototoxic conditions (solar dermatoses), hairloss, or conditions wherein an analgetic, anti-aging antiperspirant, anti-pruritic or astringent agent is helpful, comprising combining an effective amount of a tetracyclic compound, and a pharmaceutically active compound selected from the group consisting of an anti-inflammatory agent, anti-acne agent, antimicrobial, anti-hyperproliferative agent, anti-phototoxic agent, anti-hairloss agent, analgetic, anti-aging agent, antiperspirant, anti-pruritic and astringent.

17. A tetracyclic compound for use in the enhancement of skin permeability for a compound of formula I or I_{P} as defined in claim 14.

18. A tetracyclic compound for use or in a method according to claim 1, wherein the tetracyclic compound is a steroid.

19. A tetracyclic compound for use according to claim 18, wherein a tetracyclic compound is a compound having as a part of its chemical structure a group of formula 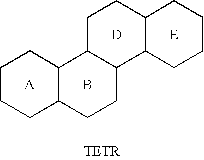
wherein each of the rings A, B, C and D have from 5 to 7 ring members.

20. A tetracyclic compound for use according to claim 19, wherein a tetracyclic compound is a corticoid.

21. A tetracyclic compound for use according to claim 18, wherein a tetracyclic compound is selected from the group consisting of aclomethasone, beclomethasone, betamethasone, clobetasole, hydrocortisone, dexamethasone, difluocortolene, fluticasone, halobetasol, halomethasone, mometasone and triamcinolone.

22. A method or use according to claim 4, wherein in a first step a tetracyclic compound and in a second step a pharmaceutically active compound is administered.
